(19) United States
(12) Patent Application Publication
(10) Pub. No.: US 2026/0199141 A1
(43) Pub. Date: Jul. 16, 2026

(54) PRESSURE-RELIEVING ATHLETIC SOCK

(71) Applicant: Patrick Emerson, Darby, PA (US)

(72) Inventor: Patrick Emerson, Darby, PA (US)

(21) Appl. No.: 19/025,171

(22) Filed: Jan. 16, 2025

(51) Int. Cl. A61F 13/08

(52) U.S. Cl. CPC A61F 13/08

(57) ABSTRACT

A pressure-relieving athletic sock includes a foot portion, a leg portion, and a cuff portion. The foot portion is designed to be placed around a foot. The foot portion includes a toe section that is designed to be placed around toes and a heel section is designed to be placed over a heel. The leg portion is attached to the foot portion adjacent the heel section. The leg portion is designed to be placed around a lower leg. The leg portion includes a front section that is designed to be placed over a shin and a back section that is designed to be placed over a calf. The leg portion includes an orifice positioned in the back section to relieve compressive force on the calf. The cuff portion is attached to the leg portion.

### (b) CROSS-REFERENCE TO RELATED APPLICATIONS

Not Applicable

### (c) STATEMENT REGARDING FEDERALLY SPONSORED RESEARCH OR DEVELOPMENT

Not Applicable

### (d) THE NAMES OF THE PARTIES TO A JOINT RESEARCH AGREEMENT

Not Applicable

### (e) INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### (f) STATEMENT REGARDING PRIOR DISCLOSURES BY THE INVENTOR OR JOINT INVENTOR

Not Applicable

### (g) BACKGROUND OF THE INVENTION

### (1) Field of the Invention

The disclosure relates to athletic footwear and more particularly pertains to a new pressure-relieving athletic sock for helping to relieve pressure on a calf of a wearer. For many athletes, muscles cramps and strains can be a problem during play of a sport, such as soccer or football. The pressure-relieving athletic sock includes openings to relieve pressure on the calf to help avoid cramps and strains.

### (2) Description of Related Art including information disclosed under 37 CFR 1.97 and 1.98

The prior art relates to socks for general use and for sports. The prior art, as best understood, does not disclose an athletic sock that includes one or more orifices at the calf area of the wearer to relieve pressure on the calf.

### (h) BRIEF SUMMARY OF THE INVENTION

An embodiment of the disclosure meets the needs presented above in a pressure-relieving athletic sock generally comprising a foot portion, a leg portion, and a cuff portion. The foot portion is designed to be placed around a foot. The foot portion includes a toe section that is designed to be placed around toes and a heel section is designed to be placed over a heel. The leg portion is attached to the foot portion adjacent the heel section. The leg portion is designed to be placed around a lower leg. The leg portion includes a front section that is designed to be placed over a shin and a back section that is designed to be placed over a calf. The leg portion includes an orifice positioned in the back section to relieve compressive force on the calf. The cuff portion is attached to the leg portion.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### (i) BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a side view of a pressure-relieving athletic sock according to an embodiment of the disclosure.

FIG. 2 is a rear view of an embodiment of the disclosure.

FIG. 3 is a side view of an embodiment of the disclosure in use.

FIG. 4 is a rear view of an embodiment of the disclosure in use.

FIG. 5 is a rear view of a first alternative embodiment of the disclosure.

FIG. 6 is a rear view of a second alternative embodiment of the disclosure.

### (j) DETAILED DESCRIPTION OF THE INVENTION

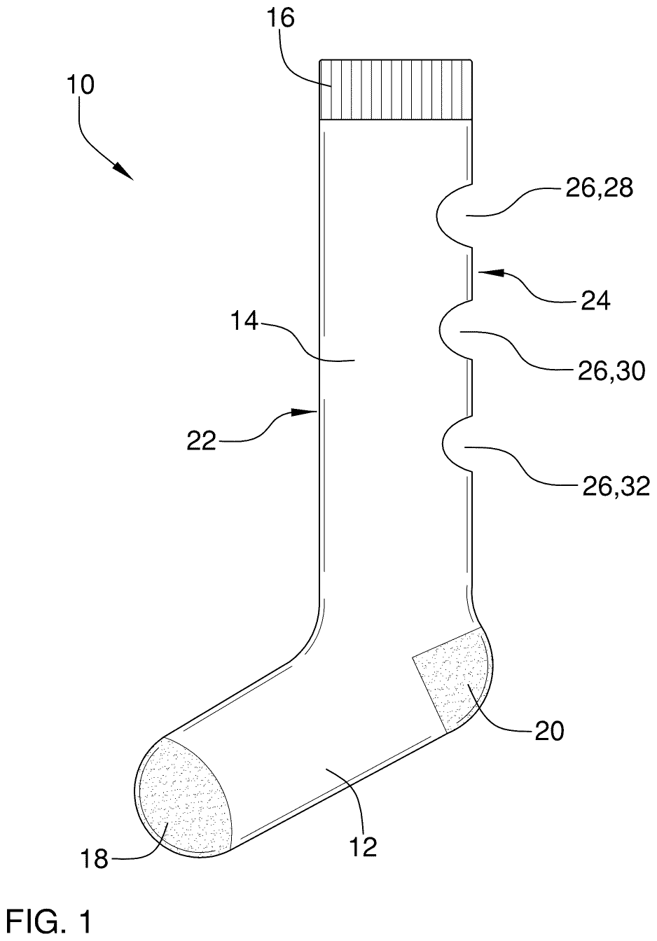

With reference now to the drawings, and in particular to FIGS. 1 through 6 thereof, a new pressure-relieving athletic sock embodying the principles and concepts of an embodiment of the disclosure and generally designated by the reference numeral 10 will be described.

As best illustrated in FIGS. 1 through 6, the pressure-relieving athletic sock 10 generally comprises a foot portion 12, a leg portion 14, and a cuff portion 16. The foot portion 12 is designed to be placed around a foot. The foot portion 12 includes a toe section 18 that is designed to be placed around toes and a heel section 20 is designed to be placed over a heel. The leg portion 14 is attached to the foot portion 12 adjacent the heel section 20. The leg portion 14 is designed to be placed around a lower leg. The leg portion 14 includes a front section 22 that is designed to be placed over a shin and a back section 24 that is designed to be placed over a calf. The leg portion 14 includes an orifice 26 positioned in the back section 24 to relieve compressive force on the calf. The cuff portion 16 is attached to the leg portion 14.

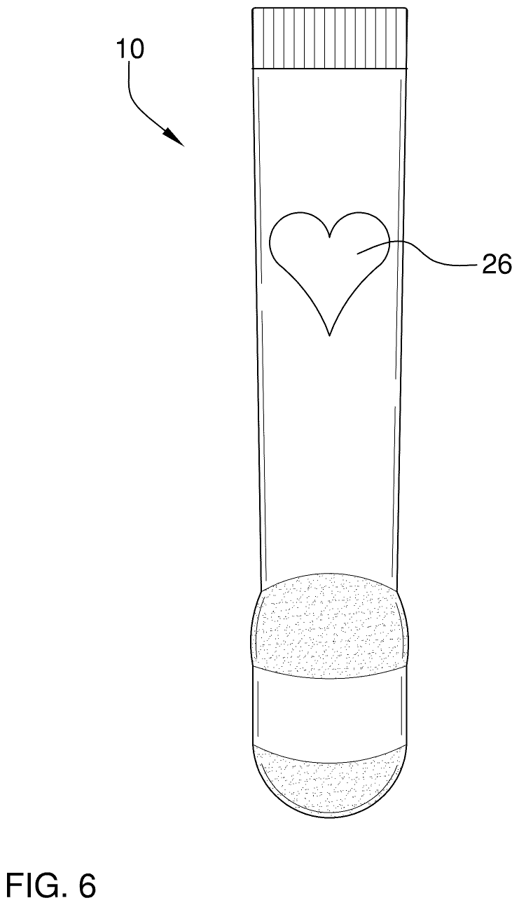

FIG. 6 shows that in one embodiment the pressure-relieving athletic sock 10 can include only one orifice 26. However, in the exemplary embodiment in FIGS. 1 through 4, the pressure-relieving athletic sock 10 includes a first orifice 28, a second orifice 30, and a third orifice 32. In one possible embodiment, there could be only the first orifice 28 and the second orifice 30, or in another possible embodiment there could be additional orifices 26 beyond the first orifice 28, the second orifice 30, and the third orifice 32. The first orifice 28 and the second orifice 30 are aligned in a vertically stacked position between the cuff portion 16 and the heel section 20 of the foot portion 12. The third orifice 32 is also aligned with the first orifice 28 and the second orifice 30 in the vertically stacked position between the cuff portion 16 and the heel section 20 of the foot portion 12.

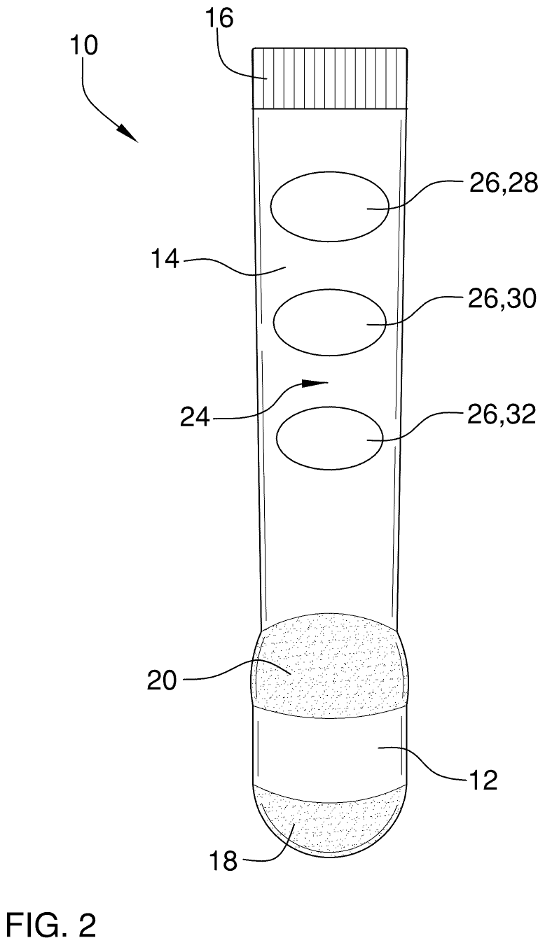

As shown in FIGS. 1 and 2, each of the first orifice 28, the second orifice 30, and the third orifice 32 is the same size. However, in another possible embodiment, each could be a different size.

In the exemplary embodiment in FIGS. 1 through 4, each of the first orifice 28, the second orifice 30, and the third orifice 32 has a geometric shape. In the exemplary embodiment in FIG. 2, the geometric shape is an oval having a length and a width. Each of the first orifice 28, the second orifice 30, and the third orifice 32 is positioned such that the length of the oval is perpendicular to a longitudinal axis of the leg portion 14. While an oval is shown, other geometric shapes, such as a circle, a triangle, or a rectangle, are within the scope of the disclosure.

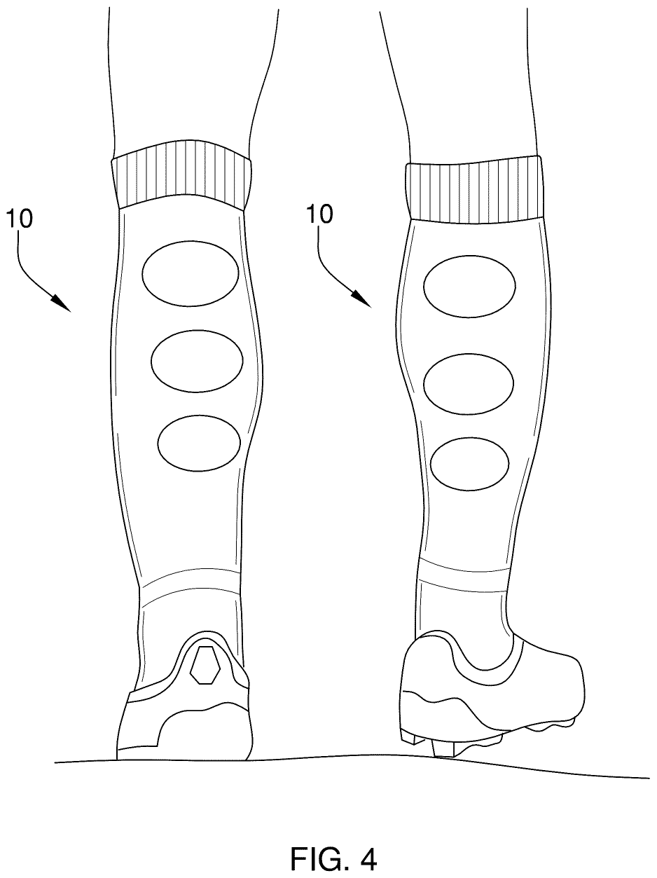

FIG. 4 shows an athlete wearing a pair of the pressure-relieving athletic socks 10. The first orifice 28, the second orifice 30, and the third orifice 32 provide pressure relief of the calf muscles.

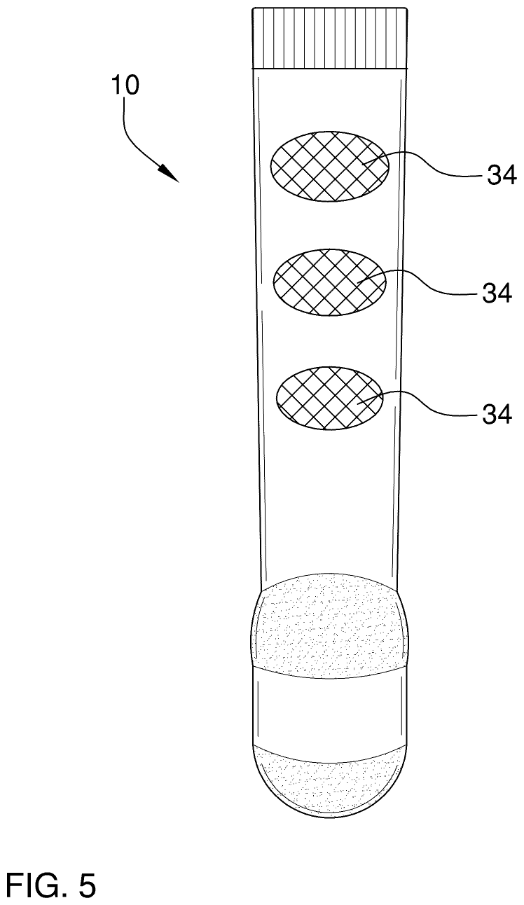

In the first alternative embodiment shown in FIG. 5, a mesh layer 34 is included that extends across each of the first orifice 28, the second orifice 30, and the third orifice 32. The mesh layer 34 provides reduced pressure but still maintains some compressive force. In one possible embodiment, reinforcing stitching or other materials, such as a resilient material, could be provided around the first orifice 28, the second orifice 30, and the third orifice 32 to prevent tearing.

In the second alternative embodiment shown in FIG. 6, the orifice 26 has a symbolic shape. The symbolic shape in this case is a heart. However, other symbolic shapes, such as currency symbols, peace signs, and animal outlines, for example, are within the scope of the disclosure.

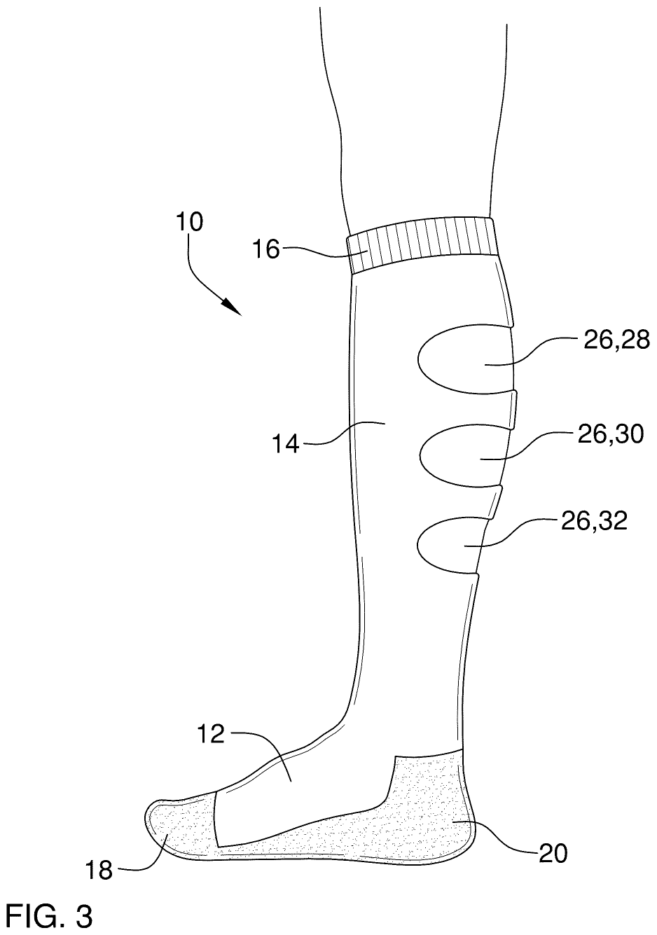

In one possible embodiment the cuff portion 16 could be omitted. In another possible embodiment, the toe section 18 and the heel section 20 could be made of different types of material than the rest of the pressure-relieving athletic sock 10. In that regard, the pressure-relieving athletic sock 10 could be made of natural or synthetic fibers, such as cotton or polyester. In yet another possible embodiment, the toe section 18 and the heel section 20 could be connected together, as shown in FIG. 3.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A pressure-relieving athletic sock comprising:
a foot portion being configured to be placed around a foot, said foot portion including a toe section being configured to be placed around toes and a heel section being configured to be placed over a heel;
a leg portion being attached to said foot portion adjacent said heel section, said leg portion being configured to be placed around a lower leg, said leg portion including a front section being configured to be placed over a shin and a back section being configured to be placed over a calf, said leg portion including an orifice positioned in said back section to relieve compressive force on the calf; and
a cuff portion being attached to said leg portion;
wherein said orifice is a first orifice and said leg portion includes a second orifice in said back section and a third orifice in said back section, wherein said first orifice, said second orifice, and said third orifice are aligned in a vertically stacked position between said cuff portion and said heel section of said foot portion, each of said first orifice, said second orifice, and said third orifice being open and equal in size and shape.

2. (canceled)

3. (canceled)

4. (canceled)

5. (canceled)

6. The pressure-relieving athletic sock of claim 1, wherein each of the first orifice, the second orifice, and the third orifice has a geometric shape.

7. The pressure-relieving athletic sock of claim 6, wherein said geometric shape of each of said first orifice, said second orifice, and said third orifice is an oval having a length and a width, wherein each of said orifices is positioned such that the length of said oval is perpendicular to a longitudinal axis of said leg portion.

8. The pressure-relieving athletic sock of claim 1, wherein each of said first orifice, said second orifice, and said third orifice has a symbolic shape.

9. The pressure-relieving athletic sock of claim 8, wherein said symbolic shape of each of said first orifice, said second orifice, and said third orifice is a heart.

10. (canceled)

11. (canceled)

12. (canceled)

13. (canceled)

14. (canceled)

15. (canceled)
